(19) United States
(12) Patent Application Publication
(10) Pub. No.: US 2024/0091557 A1
(43) Pub. Date: Mar. 21, 2024

(54) PATIENT-SPECIFIC BRACHYTHERAPY APPLICATORS

(71) Applicant: THE JOHNS HOPKINS UNIVERSITY, Baltimore, MD (US)

(72) Inventors: Marc Morcos, Baltimore, MD (US); Akila Viswanathan, Baltimore, MD (US)

(21) Appl. No.: 18/262,536

(22) PCT Filed: Jan. 21, 2022
(86) PCT No.: PCT/US2022/013334

§ 371 Date: Jul. 21, 2023

Related U.S. Application Data
(60) Provisional application No. 63/139,961, filed on Jan. 21, 2021.

(51) Int. Cl. A61N 5/10

(52) U.S. Cl. CPC A61N 5/1007; A61N 5/1016; A61N 5/1027; A61N 5/1049; A61N 2005/1005; A61N 2005/1094

(57) ABSTRACT

A design, quality assurance, and clinical use procedures are provided for real-time tracking of a custom MRI/CT-compatible brachytherapy applicators. A real-time tracking system is used for applicator implantation, repositioning and tracking during treatment delivery.

### CROSS REFERENCE TO RELATED APPLICATIONS

This application claims the benefit of U.S. Provisional Patent Application No. 63/139,961 filed on Jan. 21, 2021, which is incorporated by reference, herein, in its entirety.

### FIELD OF THE INVENTION

The present invention relates generally to medical devices. More particularly, the present invention relates to patient-specific brachytherapy applicators.

### BACKGROUND OF THE INVENTION

Brachytherapy is a radiotherapy modality which uses a small radiation source implanted into or near the tumor. Brachytherapy applicators guide the radiation source to the desired location within the patient. Intracavitary applicators are placed in naturally occurring cavities in the body such as the vagina, cervix, uterus, rectum etc. Interstitial applicators guide the radiation source through hollow needles to access deeper tumors. A common problem is that commercially available applicators are generic in shape and are not tailor-made to the patient's anatomy or tumor topology. For example, children with vaginal rhabdomyosarcoma are typically treated with intracavitary brachytherapy after surgery and chemotherapy using a vaginal cylinder applicator. However, commercially available vaginal cylinders are sized for adults, and they are difficult to use in pediatric cases given the size and inability to dose optimize away from critical pediatric structures, including the cervix and ovaries with standard applicators.

Compared to single-channel vaginal cylinders (SCVCs), multi-channel vaginal cylinders (MCVCs) improve target coverage and organ-at-risk (OAR) sparing thanks to the increased dose modulation capacity provided by the peripheral channels. The ability to adequately shape the dose distribution is of paramount importance in the setting of conformal brachytherapy. Commercially available MCVCs come in sizes which are not acceptable for pediatric use (25 to 40 mm diameter). To treat these pediatric patients, others have reported on the use of custom vaginal molds. Vaginal mold applicators are patient-specific but can lack the ability to reproducibly distend the vagina and enable uniform dose to the mucosa.

The advent of image-guided gynecologic brachytherapy has been shown to improve local control and reduce toxicities. Recent studies underline the value of MR-guidance by showing that the most important source of uncertainty in the image-guided procedure is target delineation. Therefore, applicators must be MR-compatible. Due to the hypointense signal produced in MR imaging, treatment planning including the registration a computerized applicator model to define the dwell positions is required. In the case of an applicator with an outer cylindrical symmetry such as the MCVC, careful steps must be taken to properly define the rotational alignment of the applicator. The inability to precisely and reproducibly place the applicator can cause the radiation dose the patient receives to differ from the planned dose.

Therefore, it would be advantageous to provide a device and method for image-guided, patient-specific brachytherapy applicators.

### SUMMARY OF THE INVENTION

The foregoing needs are met, to a great extent, by the present invention, wherein in one aspect a device for delivery of radiation therapy includes a brachytherapy applicator. The applicator includes a housing, and the housing defines a channel configured to receive the source of radiation. The applicator and the channel are configured and customized to provide the radiation therapy in accordance with a treatment plan for a specific patient.

In accordance with an aspect of the present invention, the radiation source includes a number of catheters configured to deliver radiation. The channel includes a single central channel in some embodiments and in others the channel includes a number of peripheral channels. A radiation shield is disposed within the applicator. The applicator takes the form of an intracavitary or interstitial (vaginal, uterine, endorectal, etc . . . ) shape. The device can also include a real-time tracking system which tracks the device (1) in the 3D coordinate system of the procedure room, and (2) in the 3D coordinate system of the patient diagnostic image series (CT, MM, US). The device includes fiducial markers. The housing is formed using 3D printing and the shape and size are based on a specific patient and treatment plan. The housing can be formed from Dental-LT.

In accordance with another aspect of the present invention, a system for delivery of radiation therapy includes an applicator. The applicator includes a housing. The housing defines channels configured to guide brachytherapy catheters and receive a source of radiation. The housing also defines identification chambers. The system includes shielding configured to protect a recipient from radiation from the source of radiation. The applicator, the channel, and the shielding are configured and customized to provide the radiation therapy in accordance with imaging (MRI, CT, US, X-ray, RGB-D) and radiation treatment plan and wherein the channels and shielding are placed in a customized configuration based on the radiation treatment plan. The system also includes a real-time tracking system that includes a tracking device. The tracking system includes imaging markers disposed in the applicator to localize the applicator for image-based treatment planning and for image-guided treatment delivery. The system also includes localizing landmarks.

In accordance with still another aspect of the present invention, the housing includes a central channel. The channel includes a number of peripheral channels. The channel takes the form of a central channel and a number of peripheral channels. The channel and catheters are secured in place relative to the applicator using a catheter locking mechanism. The applicator takes the form of an intracavitary shape (vaginal cylinder, tandem and ring, endorectal mold, . . . ). The applicator takes the form of a hybrid interstitial shape, which guides interstitial catheters to their desired location in tissue. The channel configurations are optimized to guide catheters into the tumor and away from healthy tissues in the case of interstitial implants using patient-specific imaging of anatomy and tumor topology. The channel configurations are positioned a specific distance into the applicator housing to avoid hotspots in mucosal surfaces in the case of intracavitary implants. The real-time tracking system is an optical tracking system. The applicator is tracked in the three-dimensional (3D) space of a procedure room coordinate system to track the treatment device in the room and within a 3D imaging space. The applicator is tracked in the patient image coordinate system to track the treatment device in the image (US, MRI, CT, . . . ) enabling on the fly adjustment of the implant with real-time feedback of the position relative to the tumor and organs at risk. The designed or modified medical device used has known CAD model which contains the exact geometry and dimensions of the applicator in addition to the exact locations of the tracker(s) and localization landmarks. The tracking markers are detectable using one of a combination of tracking methods such as optical, MRI active tracking, ultra-wide band radar, LIDAR. The localizing landmarks are visible on medical imaging comprising one of more of an MRI, CT, Ultrasound, RGB-D, LIDAR, SPECT or PET scan.

In accordance with still another aspect of the present invention, the system includes an intra-procedural room monitor and a computer for visualization of the tracked applicator and information on the position of the applicator. The computer receives tracking information from the tracking system and new or existing medical image scans of the patient. The computer is programmed to solve a mapping transformation between the CAD model and the tracking markers and localizing landmarks. The in-room monitor displays an overlay of the CAD model of the implanted medical device onto the medical imaging scan in real-time at regular intervals. The computer is programmed to determine location and orientation of the applicator in the imaging/patient coordinate system by mathematically solving the transformation, which maps the tracking markers to the landmarks. Alternately, the computer and monitor can take the form of one computing device. The computer can take the form of any suitable processor known to or conceivable by one of skill in the art.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A-1C illustrate views of an exemplary 3D brachytherapy applicator, according to an embodiment of the present invention.

FIG. 2 illustrates a perspective view of the constructed device in an exemplary pediatric case according to an embodiment of the present invention.

FIG. 3A illustrates a side, diagrammatic view of the components of a brachytherapy applicator, according to an embodiment of the present invention. FIG. 3B illustrates a partially sectional view of an applicator disposed within a body cavity, according to an embodiment of the present invention.

FIGS. 4A and 4B illustrate perspective and partially sectional views of an applicator configured for cervical treatment.

FIG. 5 illustrates perspective and image views of real-time tracking of the device, according to an embodiment of the present invention.

FIG. 6 illustrates graphical views of Hounsfield Unit histograms for the Dental-LT applicator (light grey) and the background Solid-Water (dark grey) are shown in the plots above where the low (1.3 mGy) and high (67 mGy) dose helical CT scans are on left and right, respectively.

FIG. 7 illustrate a graphical view of unnormalized percent-depth-dose using GafChromic-EBT3 film in Dental-LT (dark grey) and liquid water (light grey).

FIG. 8 illustrates an MR-based (T2-SPACE) treatment plan and resulting dose-volume-histogram.

FIG. 9 illustrates image views of applicator adjustment based on imaging and guided by real-time tracking.

FIG. 10 illustrates image views of an applicator with CT/MRI markers/fiducials according to an embodiment of the present invention disposed within a body cavity.

FIG. 11 illustrates views of Monte Carlo simulation results for the apex shielding, according to an embodiment of the present invention.

FIG. 12 illustrates a view of a dose ratio with and without shielding which highlights the dose reduction achieved with shielding.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides design, quality assurance and clinical use procedures for a custom MR/CT-compatible MCVC. The MCVC is made of an FDA-approved, biocompatible material. A real-time optical tracking system is used for applicator repositioning and tracking during treatment delivery.

High-dose-rate brachytherapy, also known as internal-radiation, is a radiation therapy modality in the treatment of cancer. A radioactive source (referred to herein simple as, “source”) is placed in or near the tumor. Generally, the source is a highly active radioactive source. In a preferred embodiment, the source can be 4 mm×1 mm wide. The source is welded to a cable, in some embodiments a steel cable, and the position of the source is robotically controlled to deliver the tumoricidal dose distribution. The source does not come into direct contact with the patient tissue. Instead, the source travels within implanted devices known as brachytherapy applicators.

Dosing is based on the planned dose distribution for the radiation treatment. The source sits in different pre-programmed locations for varying amounts of time. The longer a source dwells in tissue, the higher the radiation dose that is delivered to that tissue. The dose distribution designed to provide a maximum dose of radiation to the tumor and minimum damage to organs-at-risk. The source travels within the applicator to deliver the dose to the tumor. In this way, the source can be applied to different tissue regions with the appropriate dosing.

Generally, in the treatment of a body region with a natural cavity (i.e. cervix, uterine, vagina, rectum, etc.), a commercially available intracavitary applicator is placed. These applicators are not specific to the patient's personal anatomy/tumor. SCVC and MCVC brachytherapy applicators used in the treatment of endometrial and vaginal cancers are commercially available in sizes adequate for adults. Published solutions for pediatric cases include custom vaginal molds and custom SCVCs machined to a smaller diameter. MCVCs offer two major advantages over SCVCs: (1) the peripheral channels can modulate the dose distribution and shape it to achieve high tumor conformality and OAR sparing; (2) the peripheral channels are closer to the target leading to treatment plans with comparatively lower total reference air kerma (TRAK) and therefore lower total irradiated volume. Vaginal molds lack the ability to reproducibly distend the vagina enabling uniform dose to the entire vaginal (IR-CTV) mucosa. Furthermore, a case of a vagina mold tearing the vaginal opening has been reported due to the larger diameter of the impression at the apex.

Using biocompatible & sterilizable 3D-printed materials, patient-specific applicators can be produced which:
(1) Better conform to the patient
(2) Can contain high density metals to protect sensitive organs at risk
(3) Patient-specific benefits aside, these 3D-printed applicators can be produced at a fraction of the cost of commercially available applicators.
(4) The applicator prototypes have been 3D-printed with MRI fiducials so that CT is not required for treatment planning, thereby minimizing a procedure (CT scan) and reducing extra radiation dose (CT scan) to the patient.
(5) For advanced cancers requiring interstitial needle applicators, the 3D printed designs of the present invention can be designed to serve as surgical guides to guide the needle to the tumor site while avoiding organs-at-risk and blood vessels.

In an exemplary implementation of the present invention, a patient-specific 3D applicator was designed to treat a pediatric patient presenting with a lesion vaginally. The 3D applicator was designed because commercially available applicators are not available for toddlers and are too large. These young patients are typically treated with a custom applicator as the available sizes are too large. In the past, some patients were treated with devices made from dental molds made of the patient's vagina to use as an applicator. These molds were not consistent, are difficult to reproduce, and are no longer used.

Currently, treatment of pediatric patients instead uses a clinical applicator which is machined to a small diameter. In contrast, a patient-specific applicator allows for more targeted treatment and additional protection of organs-at-risk. In this exemplary instance, the patient-specific applicator was (1) designed to fit in a toddler; (2) was designed with MRI fiducial pockets for MRI visualization (since plastic-like materials are invisible in MRI); and (3) multiple strategic source channels were designed to optimize the dose distribution to the patient, which greatly reduced (up to 40%) the dose to the patient's healthy bladder, rectum, sigmoid, bowel and importantly spared the uninvolved vagina/uterus/cervix and radiosensitive ovaries.

The design of the present invention is compatible with brachytherapy treatment delivery machines. In the era of personalized and precision medicine, 3D printing is starting to be adopted by the radiation oncology community. The need for custom, patient-specific devices coupled with novel materials which meet the United States Pharmacopeia (USP) and ISO-10993 international standards for biocompatibility make 3D printing an attractive solution in a technically proficient medical specialty.

In an exemplary implementation of the invention, a 4-year-old presented with vaginal bleeding and was found on speculum examination to have a pedunculated 2 cm lesion on a stalk. Surgical resection was performed. She received chemotherapy with VAC-IE for 13 cycles and presented for brachytherapy. The brachytherapy applicator was inserted after inserting gel into the 4 marker holes embedded in the resin. 3D, T2-weighted magnetic resonance (MR) imaging was performed. These confirmed adequate depth and correct rotation. Contours included the defined post-operative bed evaluated in conjunction with the surgeon, defined as the high-risk clinical target volume (CTV_{HR}), and the remaining vaginal tissue was defined the intermediate-risk target volume (CTV_{HR}). After completion of an approved treatment plan, using daily sedation with propofol, the applicator was inserted with an optical tracking device attached. The patient was then brought into the HDR suite for treatment delivery.

Institutional approval was granted to print and use a custom 3D printed brachytherapy applicator. The applicator was 3D printed using the biocompatible Dental-LT resin (Formlabs Inc., Sommerville, MA, USA). Based on consultation with the patient's surgeon and post-operative MRI of the patient, a 14 mm diameter multichannel vaginal cylinder with four peripheral channels was designed in a CAD software (Fusion 360 Autodesk, San Rafael, CA). The applicator was designed with four small (3 mm³) pockets along the outer surface which serve as MR/CT fiducials for localizing the applicator. As the applicator appears as a hypointense signal on T2-weighted MRI, the pockets were filled with lubricant gel, known to appear markedly hyperintense, and sealed with sterile tape. An optical marker was mounted on the proximal/external portion of the applicator for real-time tracking.

The multichannel holes were made large enough to receive 2 mm diameter (6 French) rounded brachytherapy catheters. To fix the catheters in-place, the applicator was designed to use the Venezia guide tubes (Elekta Brachy, Veenendaal, The Netherlands) which have a catheter locking mechanism and repurposed them to hold catheters firmly in place within the custom applicator. To evaluate the Dental-LT resin material for brachytherapy dosimetry, Iridium-192 percent-depth-dose (PDD) in Dental-LT was measured with GafChromic-EBT3 (ISP, Wayne, NJ) films and compared to water using the film calibration protocol.

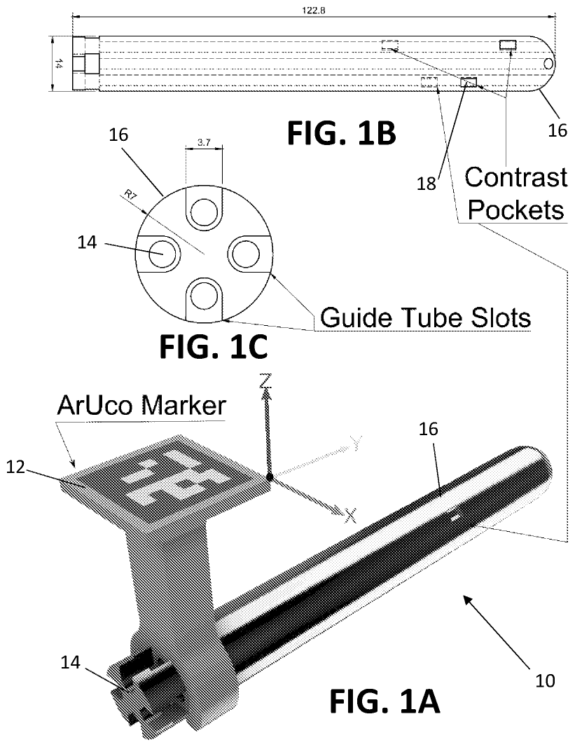

FIGS. 1A-1C illustrate views of an exemplary 3D brachytherapy applicator, according to an embodiment of the present invention. FIG. 1A illustrates a perspective view of the 3D brachytherapy applicator 10 having a real time tracking marker 12. FIG. 1B illustrates a side, semi-sectional view of the applicator 10, showing the peripheral channels 14 defined by housing 16. FIG. 1C illustrates an end view of the applicator 10 with peripheral channels 14. In this embodiment, the multi-channel applicator is designed using 14 mm diameter channels. The channels are positioned to maximize treatment benefit based on imaging for this specific patient.

An important consideration for peripheral channel configuration in the case of intracavitary applicators is that they not be too close to the mucosal surface to avoid unnecessary radiation side effect due to hotspots. In this case, a dosimetric calculation should be performed to determine the mucosal surface dose as a function of peripheral channel distance from the applicator surface.

The applicator 10 also includes four small pockets 18 that serve as MR/CT fiducials for localizing the applicator 10 for planning and real-time image-guidance. As will be described further herein, brachytherapy sources, such as catheters can be inserted into the peripheral channels 14. In an exemplary embodiment, the real-time tracker takes the form of an ArUco Marker real-time tracking jig.

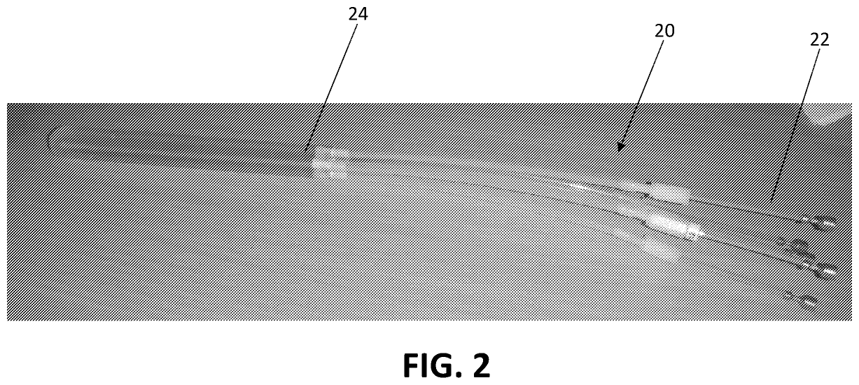

FIG. 2 illustrates a perspective view of a source to be disposed within the brachytherapy applicator, according to an embodiment of the present invention. The source 20 includes catheters 22 that diverge from hub 24. Each catheter 22 is configured to apply radiation. Four catheters 22 are shown in FIG. 2 with respect to the embodiment described herein. However, this embodiment is provided by way of example and should not be considered limiting. Any number of catheters 22 determined for the treatment can be used.

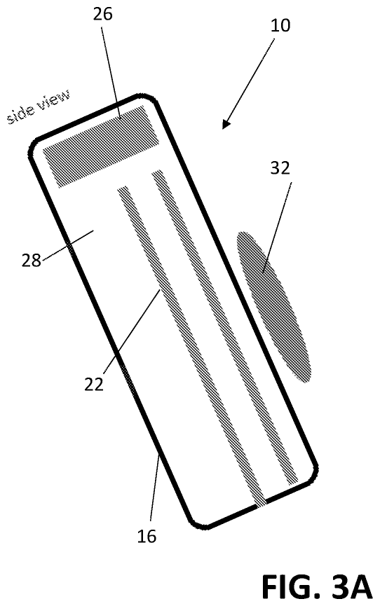

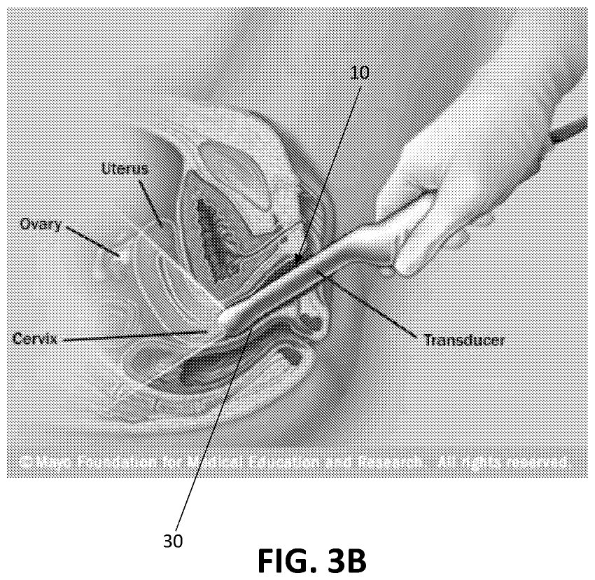

FIG. 3A illustrates a side, diagram view of the components of a brachytherapy applicator, according to an embodiment of the present invention. FIG. 3B illustrates a partially sectional view of an applicator disposed within a body cavity, according to an embodiment of the present invention. The applicator 10 includes a housing 16. A shield 26 can be disposed within a space 28 created by housing 16. While the shield is positioned at a distal end of the applicator, as shown in FIG. 3A, the shielding can be positioned, as required by the treatment plan. The shield 26 is used to protect organs-at-risk or other sensitive anatomical structures. The catheters 22 are also disposed within the housing 16. The applicator 10 is configured to be positioned within a body cavity 30. As illustrated in FIG. 3A, the applicator 10 can be positioned adjacent to tumor 32. The design and configuration of the applicator 10 also allows it to be repositioned to complete treatment from various angles and at various levels of radiation as determined by the treatment team.

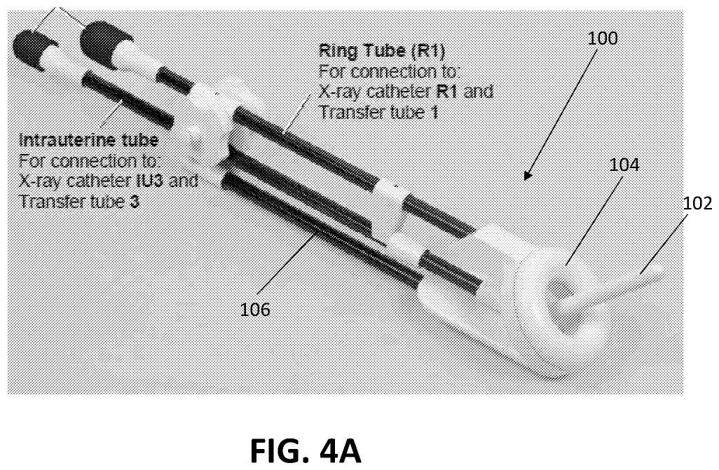

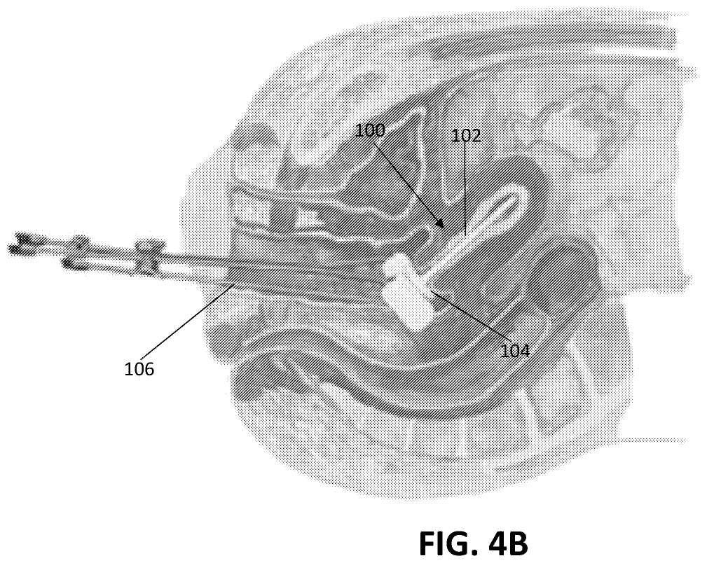

FIGS. 4A and 4B illustrate perspective and partially sectional views of an applicator configured for cervical treatment. The applicator 100 includes a tandem and ring configuration. The tandem 102 is configured to be disposed within the cervix or other luminal space. The ring 104 is disposed at the base of the tandem and can be positioned at the cervix or other relevant physical structure. Tubes 106 are coupled to the tandem and ring applicator to provide brachytherapy radiation according to a treatment plan. One exemplary tube 106 is a ring tube that is configured for connection to an x-ray catheter R1 and a transfer tube 1. Another exemplary tube 106 is an intrauterine tube for connection to an x-ray catheter IU3 and transfer tube 3. The size, diameter, and angles of the tandem, ring, and applicator can be optimized based on the patient's anatomy and treatment plan. The dwell time in each position is also dictated by the treatment plan.

In the exemplary implementation of the present invention, the patient was positioned on an MR-compatible transfer table (Zephyr XL; Diacor, Inc., Salt Lake City, UT) in an MRI simulator and brachytherapy procedure room. MR-simulation and image guidance were performed using a 3D T2-weighted turbo spin-echo with variable flip-angle sequence (T2-SPACE) acquired on a 1.5-T Magnetom Espree (Siemens Medical Solutions, Malvern, PA, USA). The approximately 7-minute acquisition has echo (TE) and repetition times (TR) of 97 and 3500 ms, respectively, and produces images with voxel dimensions of 1.0×1.0×1.6 mm³. The patient was then transferred to the HDR suite for treatment. Rigid image fusion to the public symphysis between the reference (MR-sim) and the daily image-guidance (MR or CT) was performed to evaluate applicator insertion depth and rotation.

On days when MRI was not available, applicator insertion and CT image guidance was performed in the HDR suite using a mobile CT scanner (AIRO, Brainlab Inc.). Although typical CT doses are relatively small compared to the brachytherapy treatment, it is worthwhile to minimize unwanted doses given the patient's history. A very low dose CT protocol (1.3 mGy CTDIv0116-cm; helical acquisition; 9.6 mAs, 120 kVp; standard filter; 1 mm slice thickness; 256 mm FOV) was designed based on a technical assessment. A phantom study was performed in advance to ensure that the applicator would be visible and distinguishable from the surrounding soft tissue when imaging the patient. The 20 cm diameter Gammex-464 ACR CT accreditation phantom with bone insert (Gammex, Middleton, WI, USA) was used as a patient surrogate and the applicator was placed in the air cavity.

In the context of this work, real-time tracking means the continuous tracking of an object pose in space, where the pose contains both position and orientation of the applicator with respect to the patient. A real-time optical tracking system is included as an element of the present invention. One exemplary implementation of this real-time tracking system is based on the open-source computer vision (OpenCV) library using ArUco markers. 3D pose estimation using ArUco markers can achieve sub-millimeter and sub-degree accuracy with careful calibration.

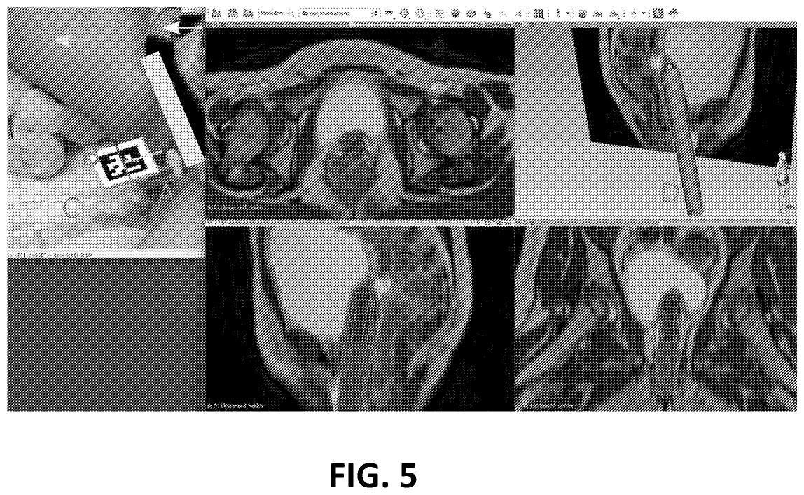

FIG. 5 illustrates perspective and image views of real-time tracking of the device, according to an embodiment of the present invention. The tracking system tracks the brachytherapy applicator and using the OpenIGTLink protocol transmits the applicator transform to 3D Slicer (Slicer v4.8.0, https://www.slicer.org). A display shows the treatment team the real-time position of the device during treatment. This display can be multimodal. A live camera feed is updated at 20-120 times per second and displays the real-time offsets to the applicator shift and rotation relative to the scan baseline. Here, a Dental-LT applicator (A) with ArUco tracking marker (B) and catheter-locking guide tubes (C) are shown in the live feed. The 3D Slicer research platform is updated by the real-time tracking system and updates the position and orientation of the applicator model (D). This positional information is overlaid over image information for accurate tracking. For the purposes of MCVC brachytherapy, the tracking system displays the shift and applicator rotation in the applicator frame of reference and long axis, respectively. Real-time tracking of the applicator is visualized in 3D slicer using the daily imaging (MR or CT). This is done by importing the 3D applicator model, registering the model to the image using the 4 fiducial pockets and solving the rigid transform and applying this transformation on top of the tracking transform.

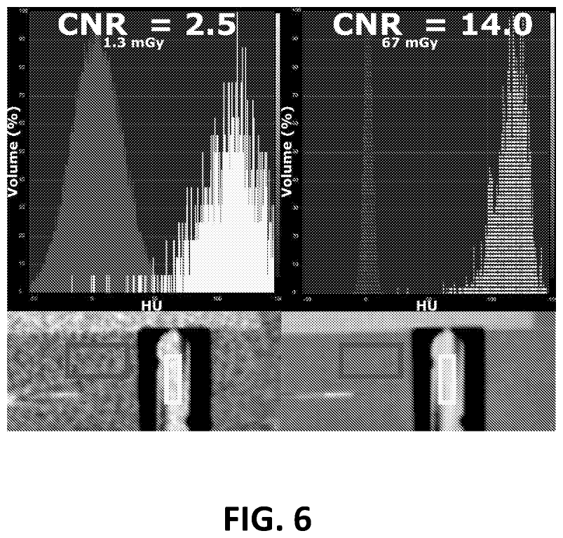

On CT imaging, the Dental-LT applicator is uniform in density with a mean Hounsfield unit of 115 HU. Using a standard pelvis CT scanning protocol (67 mGy CTDIᵥₒₗ 16-cm; 324 mAs; 120 kVp; standard filter; 1 mm slice thickness; 256 mm FOV) a contrast-to-noise ratio (CNR) of 14.0 was observed. The custom low dose protocol of the present invention has considerably more noise but enabled sufficient soft tissue to applicator discrimination with a CNR of 2.5. HU histograms for the phantom and applicator for both protocols are shown in FIG. 6. FIG. 6 illustrates graphical views of Hounsfield Unit histograms for the Dental-LT applicator (light grey) and the background Solid-Water (dark grey) are shown in the plots above where the low (1.3 mGy) and high (67 mGy) dose helical CT scans are on left and right, respectively.

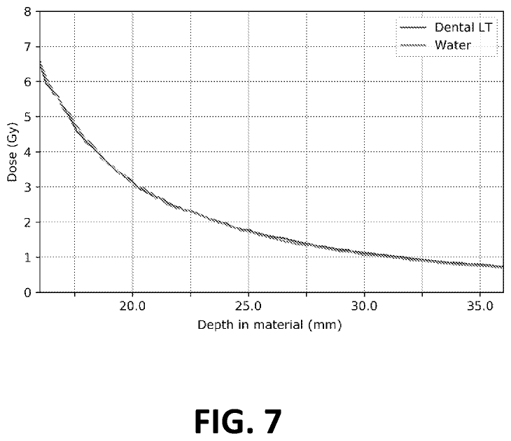

Unnormalized PDDs in Dental-LT and liquid water are shown in FIG. 7. PDDs were within 2% of each other in the evaluated depth range. Doses higher than 6 Gy were excluded as they were beyond the red-channel film calibration. FIG. 7 illustrate a graphical view of unnormalized percent-depth-dose using GafChromic-EBT3 film in Dental-LT (dark grey) and liquid water (light grey). Doses closer higher than 6 Gy were excluded as the red-channel-based film calibration was validated to 6 Gy.

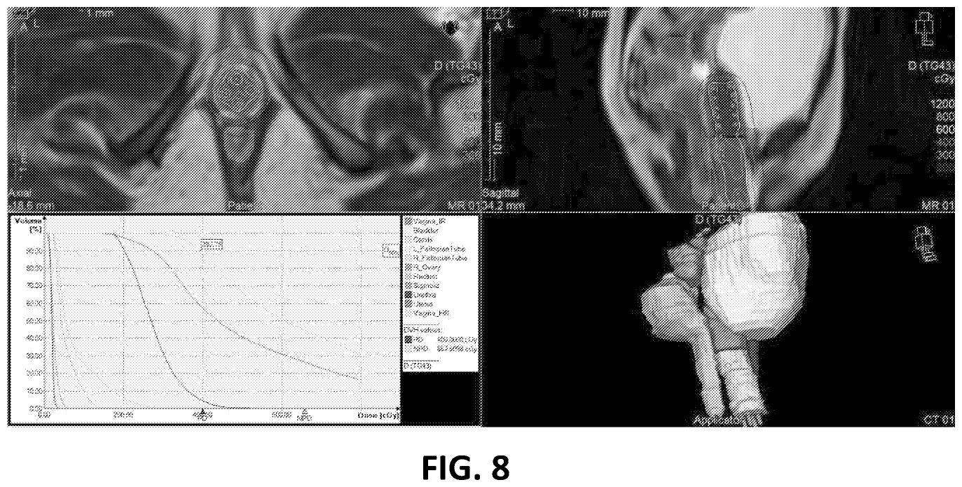

The MR-based treatment plan is shown in FIG. 8. FIG. 8 illustrates an MR-based (T2-SPACE) treatment plan and resulting dose-volume-histogram. A prescription of 4 Gy/fx and 3 Gy/fx to the high-risk and intermediate-risk vaginal CTVs, respectively, was used. Dose-volume-histogram (DVH) metrics for the MCVC and a hypothetical SCVC with equivalent diameter for the 7-fraction treatment are listed in Table 1. For equal CTVHR D90%, SCVC achieves a 16% higher dose to the CTV_{HR} at the expense of increasing all OAR doses by up to 29%. The total EQD2 doses for the MCVC is summarized in Table 2.

TABLE 1
DVH metric comparison for plans generated for MCVC and
SCVC applicators. Treatment plans were designed to deliver
4 Gy/fx to the CTVHR and up to 3 Gy/fx to the CTVIR. SCVC
plan was normalized to achieve equal CTVHR D90%.

Total Dose (Gy)

| DVH Metric | MCVC | SCVC |
|---|---|---|
| CTV_{HR} D_{90%} | 27.5 | 27.5 |
| CTV_{IR} D_{90%} | 19.0 | 22.1 |
| Bladder D_{2 cc} | 13.7 | 16.2 |
| Rectum D_{2 cc} | 14.4 | 15.6 |
| Sigmoid D_{2 cc} | 2.5 | 3.2 |
| Urethra D_{50%} | 18.8 | 21.6 |
| Uterus D_{50%} | 1.4 | 1.7 |
| Ovaries D_{50%} | 1.4 | 1.7 |
| Cervix D_{50%} | 3.1 | 4.0 |

TABLE 2
Total EQD2 doses for the entire 7 fraction treatment
using the MCVC applicator. α/β of 3 Gy and
10 Gy are used for OARs and targets, respectively.

| DVH Metric | Total EQD2 | |
|---|---|---|
| CTV_{HR} D_{90%} | 31.9 | Gy₁₀ |
| CTV_{IR} D_{90%} | 20.2 | Gy₁₀ |
| Bladder D_{2 cc} | 13.5 | Gy₃ |
| Rectum D_{2 cc} | 14.6 | Gy₃ |
| Sigmoid D_{2 cc} | 1.7 | Gy₃ |
| Urethra D_{50%} | 21.5 | Gy₃ |
| Uterus D_{50%} | 0.9 | Gy₃ |
| Ovaries D_{50%} | 0.9 | Gy₃ |
| Cervix D_{50%} | 2.1 | Gy₃ |

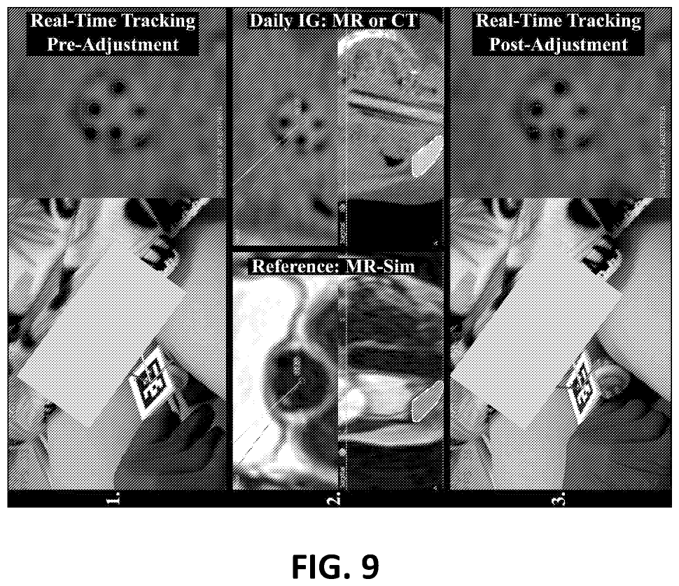

Real-time tracking, as illustrated in FIG. 9 is used to assist in repositioning the applicator to match simulation and to ensure that the applicator position does not change during treatment delivery. Applicator to DICOM rigid registration was achieved with root-mean-square error (RMSE) ranging from 0.16 to 0.64 mm for all seven fractions. FIG. 9 illustrates image views of applicator adjustment based on imaging and guided by real-time tracking. (1) Tracking based on applicator position at the time of daily imaging. (2) Rigid registration (to pubic symphysis, in white) to evaluate applicator insertion depth and rotation. (3). Tracking based on applicator position after repositioning.

Dental-LT resin is used to form the applicator described herein due to its biocompatibility, durability and the ability to 3D print this material, and because of the need to have image-guidance with both MR and CT. To implement this applicator for clinical use conducted a validation of applicator visibility on MR and CT imaging and determined the radiation attenuation properties relative to water. The novel applicator of the present invention includes embedded MR-/CT-pockets filled with lubricant gel used as imaging fiducials that enable localization of the applicator in addition to its orientation which is an important degree of freedom with MCVCs. It should be noted that while Dental-LT resin is described herein as the material used to form the applicator, this is an exemplary implementation of the invention. Other materials known to or conceivable by one of skill in the art are considered to be included within the scope of this invention.

A tuned, very low dose CT protocol was designed in conjunction with the manufacturer to meet pediatric CT imaging standards and was adequate for assessing applicator placement in 3D while reducing the additional dose burden to the patient to a minimum. Though radiation safety reports a total of 25 mGy dose for children, this methodology resulted in significantly lower dose than threshold. It was important to ensure that the applicator was water-equivalent due to the inability of the TG-43 dose formalism to handle material heterogeneities.

Optical tracking has been used extensively in neurosurgery. Tracking with low-cost, open source, optical tracking of augmented reality markers been shown to achieve submillimeter pose estimation. Granted, optical tracking requires line of sight with the tracked object, it is a robust and flexible technique which can accurately localize the position and orientation of devices in a variety of environments. Due to the large dose gradients in brachytherapy a 1 mm positioning error can result in a 20% change in dose at 1 cm from an ¹⁹²Ir source. Providing an accurate localization of the applicator is therefore crucial when repositioning the applicator to match the planned position based on image-guidance immediately prior to treatment. Furthermore, real-time tracking can ensure that the applicator remains in-place and in the correct orientation (e.g. MCVC insertion depth and rotation) during treatment delivery. To ensure reproducible treatments and patient safety, the real-time tracking system is used to accurately match daily applicator insertions to simulation and ensured stable positioning during treatment delivery.

With the patient under conscious sedation, the patient may move during treatment delivery. Brachytherapy needs to be precise due to the high dose of radiation and large dose gradients. Tracking can also reduce the need for repeat imaging to verify position of implanted applicator. A prototype system has been made which uses the treatment room's high quality cameras. The present invention thereby uses computer vision programming coupled with MM/CT image tracking. By implementing an asymmetric tracking object onto the applicator, the applicator-relative-position and orientation can be calculated in the patient's frame of reference. This allows the treatment team to determine whether the applicator has moved inside the patient, and whether the orientation is constant.

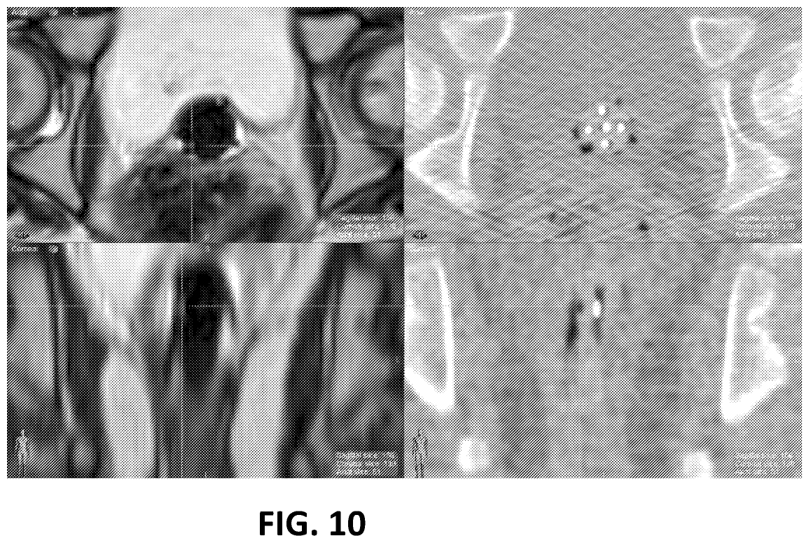

FIG. 10 illustrates image views of an applicator according to an embodiment of the present invention disposed within a body cavity. As seen in the images on the left, the applicator is invisible on MRI imaging. Pockets are added to the applicator and a syringe is used to inject MRI contrast material prior to use of the applicator. As can be seen in the images on the left, after the MRI contrast is injected the applicator can be seen the MRI images. The four fiducials shown here allow daily pre-treatment image guidance using MRI. This protocol avoids additional daily CT dose for pediatric patients.

In practice, the applicator is inserted in patient. While, the patient is on the procedure table, the patient is imaged using an imaging modality such as, but not limited to MM, CT, SPECT, or Ultrasound. The applicator device is localized using the tracking system, and the location and orientation of the applicator in the imaging/patient coordinate system is determined by mathematically solving the transformation, which maps the tracking markers to the landmarks. An overlay of the medical scan and the real-time position of the medical device are shown to the clinician or displayed on a monitor associated with the system. This enables the clinician to manipulate and move the device safely (avoids cutting important organs, or guides biopsy needle to center of tumor, etc. . . . ).

The tracking system of the device of the present invention includes a tracking device, tracking markers, and localizing landmarks. A CAD model of the applicator device being used contains the exact geometry and dimensions of the applicator in addition to the exact locations of the tracker(s) and localization landmarks. This allows for the most accurate tracking of the applicator device. The tracking markers are detectable using one of a combination of tracking methods such as optical, MRI active tracking, ultra-wide band radar, LIDAR. The localizing landmarks are visible on medical imaging including one of more of an Mill, CT, Ultrasound, RGB-D, LIDAR, SPECT or PET scan. The system further includes an intra-procedural room monitor and a computer for visualization of the tracked applicator and information on the position of the applicator. The computer receives tracking information from the tracking system and new or existing medical image scans of the patient. The computer is also programmed to solve the mapping transformation between the CAD model and the tracking markers and localizing landmarks. Further, the in-room monitor displays an overlay of the CAD model of the implanted medical device onto the medical imaging scan in real-time at regular intervals.

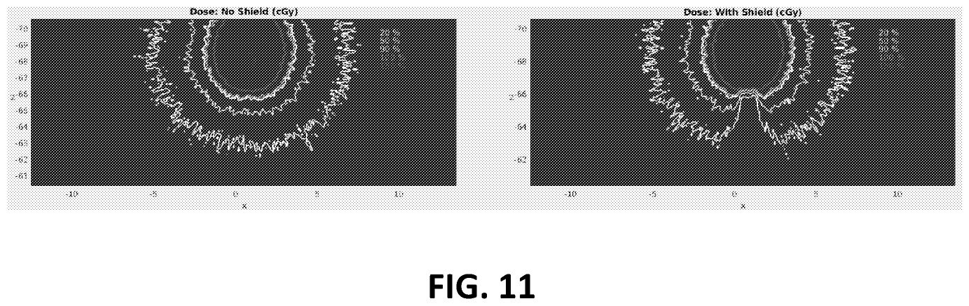

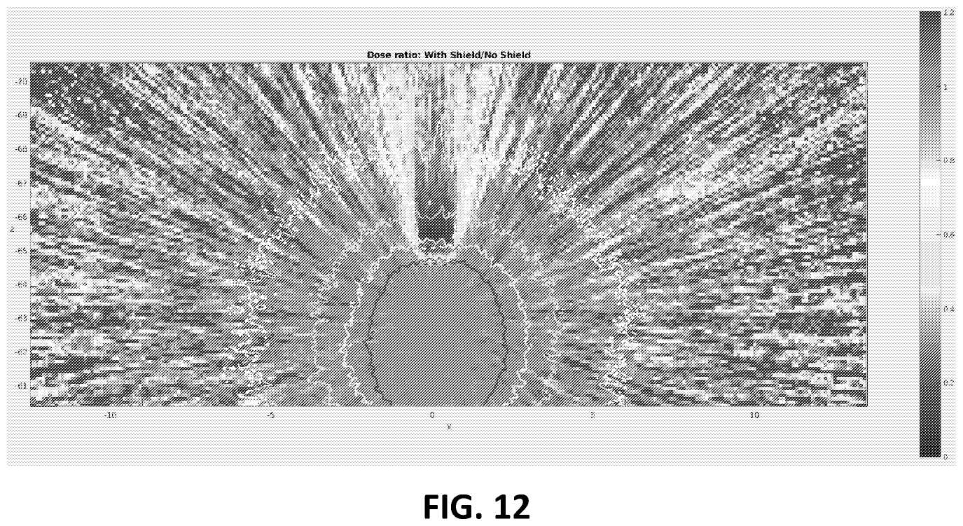

FIG. 11 illustrates views of Monte Carlo simulation results for the apex shielding, according to an embodiment of the present invention. FIG. 12 illustrates a view of a dose ration with and without shielding. The view on the left of FIG. 11 shows the dose without shielding. The view on the right of FIG. 11 shows the dose with shielding. As can be seen the shield can significantly reduce dosing for localized areas. This configuration of the applicator of the present invention provides additional protection for non-tumor tissues and OARs. Shielding might not be the right configuration for each patient. For example, shielding was not used in the exemplary implementation described herein because the very small diameter applicator required for the patient led to a very small solid angle. The tilt of the patient's uterus was out of the shielded cone. In addition, this particular patient required the entire vagina to receive a high dose with the apex included. The technique of shielding could, however, benefit a number of patients. Therefore, the ability to conform the design of the present invention to a number of patient specific treatment plans is highly desirable.

The control and real time tracking of the present invention can in some instances be carried out using a computer, non-transitory computer readable medium, or alternately a computing device or non-transitory computer readable medium incorporated into the applicator. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The computing device can be part of the Mill system or other imaging system. The Mill system is controlled by an MRI console, which controls all of the components of the system. The operating console for the device is a non-generic computer specifically designed by the manufacturer. It is not a standard business or personal computer that can be purchased at a local store. Additionally, the console computer can carry out communications through the execution of proprietary custom-built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A system for delivery of radiation therapy comprising:
an applicator, wherein the applicator comprises a housing, wherein the housing defines channels configured to guide brachytherapy catheters wherein the housing is configured to receive a source of radiation, and wherein the housing defines identification chambers;
shielding configured to protect a recipient from radiation generated by the source of radiation;
wherein the applicator, the channel, and the shielding are configured and customized to provide the radiation therapy in accordance with an imaging and radiation treatment plan and wherein the channels and shielding are placed in a customized configuration based on the radiation treatment plan;
a real-time tracking system comprising:
a tracking device;
imaging markers disposed in the applicator to localize the applicator for image-based treatment planning and for image-guided treatment delivery; and,
localizing landmarks.

2. The system of claim 1, wherein the housing comprises central channels.

3. The system of claim 1, wherein the channels comprise a number of peripheral channels.

4. The system of claim 1, wherein the channels comprise a central channel and a number of peripheral channels.

5. The system of claim 1, wherein the channels and catheters are secured in place relative to the applicator using a catheter locking mechanism.

6. The system of claim 1, wherein the applicator takes the form of an intracavitary shape.

7. The system of claim 1, wherein the applicator takes the form of a hybrid interstitial shape which guides interstitial catheters to their desired location in tissue.

8. The system of claim 1, wherein the channel configurations are optimized to guide catheters into the tumor and away from healthy tissues in the case of interstitial implants using patient-specific imaging of anatomy and tumor topology.

9. The system of claim 1, wherein the channel configurations are positioned a specific distance into the applicator housing to avoid hotspots in mucosal surfaces in the case of intracavitary implants.

10. The system of claim 1 wherein the real-time tracking system is an optical tracking system.

11. The system of claim 1, wherein the applicator is tracked in the three-dimensional (3D) space of a procedure room coordinate system to track the treatment device in the room and within a 3D imaging space.

12. The system of claim 1, wherein the applicator is tracked in the patient image coordinate system to track the treatment device in the image enabling on the fly adjustment of the implant with real-time feedback of the position relative to the tumor and organs at risk.

13. The system of claim 1, wherein the designed or modified medical device used has known CAD model which contains the exact geometry and dimensions of the applicator in addition to the exact locations of the tracker(s) and localization landmarks.

14. The system of claim 1 wherein the tracking markers are detectable using one or a combination of tracking methods such as optical, MRI active tracking, ultra-wide band radar, LIDAR.

15. The system of claim 1 wherein the localizing landmarks are visible on medical imaging comprising one of more of an MRI, CT, Ultrasound, RGB-D, LIDAR, SPECT or PET scan.

16. The system of claim 1 further comprising an intra-procedural room monitor and a computer for visualization of the tracked applicator and information on the position of the applicator.

17. The system of claim 16, wherein the computer receives tracking information from the tracking system and new or existing medical image scans of the patient.

18. The system of claim 16, wherein the computer is programmed to solve a mapping transformation between the CAD model and the tracking markers and localizing landmarks.

19. The system of claim 16, wherein the in-room monitor displays an overlay of the CAD model of the implanted medical device onto the medical imaging scan in real-time at regular intervals.

20. The system of claim 16, wherein the computer is programmed to determine location and orientation of the applicator in the imaging/patient coordinate system by mathematically solving the transformation, which maps the tracking markers to the landmarks.
